(19) United States
(12) Patent Application Publication
(10) Pub. No.: US 2020/0390679 A1
(43) Pub. Date: Dec. 17, 2020

(54) POLY(VINYLAMINE-VINYLFORMAMIDE) COPOLYMERS AS ANTIPERSPIRANT AGENT

(71) Applicant: Henkel AG & Co. KGaA, Duesseldorf (DE)

(72) Inventors: BERNHARD BANOWSKI, Duesseldorf (DE); INGA KERSTIN VOCKENROTH, Duesseldorf (DE); MARCUS CLAAS, Hilden (DE)

(73) Assignee: Henkel AG & Co. KGaA, Duesseldorf (DE)

(21) Appl. No.: 16/976,960

(22) PCT Filed: Jan. 8, 2019
(86) PCT No.: PCT/EP2019/050315

§ 371 Date: Aug. 31, 2020

(30) Foreign Application Priority Data
Mar. 1, 2018 (DE) 10 2018 203 044.8

(51) Int. Cl. A61K 8/81; A61K 8/20; A61K 8/23; A61K 8/24; A61K 8/362; A61K 8/365; A61K 8/92; A61K 8/06; A61K 8/86; A61Q 15/00

(52) U.S. Cl. CPC A61K 8/817; A61K 8/20; A61K 8/23; A61K 8/24; A61K 8/362; A61K 8/365; A61K 2800/56; A61K 8/062; A61K 8/86; A61Q 15/00; A61K 2800/30; A61K 2800/41; A61K 2800/48; A61K 8/92

(57) ABSTRACT

The present disclosure relates to an antiperspirant cosmetic composition comprising, a poly(vinylamine-vinylformamide) copolymer having a molecular weight of from about 300 to about 20,000 g/mol in a total amount of from about 0.1 to about 20% by weight, an emulsifier in a total amount of from about 0.05 to about 10% by weight, and an oil component in a total amount of from about 0.05 to about 50% by weight %. The cosmetic has a pH of from about 2.5 to about 7.5, the poly(vinylamine-vinylformamide) copolymer is neutralized or polycationic, and the cosmetic contains an anion selected from chloride, phosphates, and sulphates. The cosmetic does not contain halides and/or hydroxyhalides of aluminium and/or zirconium. These polymers make it possible to provide antiperspirant cosmetic products that do not contain aluminium salts and/or zirconium salts yet are highly effective and have less staining on textiles.

### CROSS-REFERENCE TO RELATED APPLICATION

This application is a U.S. National-Stage entry under 35 U.S.C. § 371 based on International Application No. PCT/EP2019/050315, filed Jan. 8, 2019, which was published under PCT Article 21(2) and which claims priority to German Application No. 10 2018 203 044.8, filed Mar. 1, 2018, which are all hereby incorporated in their entirety by reference.

### TECHNICAL FIELD

The present disclosure concerns an antiperspirant cosmetic composition comprising, in an aqueous cosmetic carrier, at least one poly(vinylamine-vinylformamide) copolymer present together with selected anions, the cosmetic composition having an acidic or neutral pH value. The use of such a cosmetic product makes it possible to provide antiperspirant cosmetic products which do not contain aluminium salts and/or zirconium salts yet are highly effective and have less staining on textiles.

Furthermore, the present disclosure concerns a non-therapeutic cosmetic method for preventing and/or reducing perspiration of the body, in which an antiperspirant cosmetic agent as contemplated herein is applied to the skin, in particular to the skin of the armpits, and remains on the skin of the armpits for at least about 1 hour, preferably for at least about 2 hours, preferably for at least about 4 hours, and in particular for at least about 6 hours.

### BACKGROUND

Washing, cleaning and caring for one's own body is a basic human need and modern industry is constantly trying to meet these needs in a variety of ways. Particularly important for daily hygiene is the permanent elimination or at least reduction of body odor and underarm wetness. Numerous special deodorizing or antiperspirant body care products are known in the state of the art, which have been developed for use in body regions with a high density of sweat glands, especially in the armpit area. These are available in a wide variety of dosage forms, for example as powder, in stick form, as aerosol spray, pump spray, liquid and gel roll-on application, cream, gel and as impregnated flexible substrates (deodorants).

State-of-the-art cosmetic antiperspirants contain at least one antiperspirant compound in the form of aluminium salts and/or aluminium-zirconium salts, in particular in the form of halides and/or hydroxyhalides of aluminium and/or zirconium, in addition to at least one oil or wax and a fragrance component or perfume. On the one hand, these antiperspirant compounds reduce the body's sweat secretion by temporarily narrowing and/or clogging the ducts of the sweat glands, so that the amount of sweat can be reduced by from about 20 to about 60 percent. On the other hand, they have an additional deodorizing effect due to their antimicrobial effect.

Halogenides and/or hydroxyhalides of aluminium and/or zirconium in combination with the acidic pH value of these antiperspirants may cause unpleasant skin reactions for some users. In addition, the use of the aforementioned antiperspirant compounds can lead to staining of clothing.

WO 2006/018073 A1 reveals various polyamines, including polyethyleneimines, as alternatives to halides and/or hydroxyhalides of aluminium and/or zirconium as antiperspirants. These antiperspirants are described as flocculating in WO 2006/018073 A1, which means that the light transmission of natural sweat is reduced by at least about 10% on contact with the antiperspirant. WO 2006/018073 A1 further reveals that the flocculating water-soluble polymers are partially or completely quaternized at a pH of from about 4 to about 8. It is assumed that the antiperspirant effect is based on the fact that the flocculations formed block the outlets of sweat glands. The formation of flocculation is often perceived by consumers as visually unpleasant, and the flocculation can also get into clothing and cause stains.

### BRIEF SUMMARY

The purpose of the present disclosure was to provide antiperspirants without halides and/or hydroxyhalides of aluminium and/or zirconium. The antiperspirant active substances contained in the cosmetic product should have an improved or particularly good antiperspirant effect and less staining on textiles.

It has now been surprisingly found that the use of certain poly(vinylamine-vinylformamide) copolymers contained in a cosmetic product adjusted to an acidic to neutral pH and containing certain anions has an antiperspirant effect that is even comparable to the antiperspirant effect of formulations containing aluminium salts and/or aluminium-zirconium salts. Accordingly, the antiperspirants as contemplated herein do not contain antiperspirant halides and/or hydroxyhalides of aluminium and/or zirconium.

Antiperspirant cosmetic compositions and methods for reducing body odor are provided. In an exemplary embodiment, the antiperspirant cosmetic composition includes a poly(vinylamine-vinylformamide) copolymer having a molecular weight of from about 300 to about 20,000 g/mol in a total amount of from about 0.1 to about 20% by weight. The antiperspirant cosmetic composition further includes an emulsifier in a total amount of from about 0.05 to about 10% by weight, and an oil component in a total amount of from about 0.05 to about 50% by weight. The antiperspirant cosmetic composition has a pH value of from about 2.5 to about 7.0, and the poly(vinylamine-vinylformamide) copolymer is neutralized or polycationic. The antiperspirant cosmetic composition includes anion selected from the group of chloride, phosphates, and sulphates, and is free of halides and/or hydroxyhalides of aluminum and/or zirconium. In a method of use, the antiperspirant cosmetic composition is applied to the skin and remains on the skin for at least one hour.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The subject matter of the present disclosure is antiperspirant cosmetic agents which are contained in an aqueous cosmetic carrier, and which contain, in relation to the total amount of the cosmetic agent:

a) at least one poly(vinylamine-vinylformamide) copolymer having a molecular weight of from about 300 to about 20,000 g/mol in a total amount of from about 0.1 to about 20% by weight,
b) at least one emulsifier in a total amount of from about 0.05 to about 10% by weight, and
c) at least one oil component in a total amount of from about 0.05 to about 50% by weight, where
the cosmetic composition has a pH value of from about 2.5 to about 7.5 and the poly(vinylamine-vinylformamide) copolymer is neutralized or polycationic,
the cosmetic product contains at least one anion selected from the group of chloride, phosphates and sulphates, and
the cosmetic product does not contain halides and/or hydroxyhalides of aluminium and/or zirconium.

As contemplated herein, the term “antiperspirant” is understood to mean the decrease or reduction of perspiration of the perspiratory glands of the body.

The term “halides and/or hydroxyhalides of aluminium and/or zirconium” in the context of the present disclosure means in particular chlorides, bromides and iodides of aluminium and zirconium as well as compounds of the formulae Al(OH)_{y}X and Zr(OH)_{z}X, wherein X in the aforementioned formulae stands for a halide ion. The fact that, as contemplated herein, no halides and/or hydroxyhalides of aluminium and/or zirconium are contained in the cosmetic composition means that their content is below about 0.5% by weight, preferably below about 0.1% by weight, particularly preferably 0% by weight.

Unless otherwise indicated, percentages by weight in the present disclosure refer to the total amount of the cosmetic product, excluding any propellant.

Essential for the present disclosure is the presence of at least one poly(vinylamine-vinylformamide) copolymer with a molecular weight of from about 300 to about 20,000 g/mol in a total amount of from about 0.1 to about 20% by weight. Preferred antiperspirant cosmetic compositions as contemplated herein contain from about 0.5 to about 10%, preferably from about 1.0 to about 7.0%, more preferably from about 2.0 to about 5.0%, by weight of the poly(vinylamine-vinylformamide) copolymer.

The poly(vinylamine-vinylformamide) copolymers are produced by polymerization of vinylformamide followed by partial hydrolysis of the polyvinylformamide.

It has been shown that, in the context of the present disclosure, polymers with a certain degree of hydrolysis (and thus a certain content of vinylamine monomer building blocks) are particularly effective. Preferred antiperspirant cosmetic compositions as contemplated herein include the poly(vinylamine-vinylformamide) copolymer in an amount of from about 10-99 mol %, preferably from about 25-98.5 mol %, more preferably from about 50-98 mol %, still more preferably from about 75-97.5 mol %, still more preferably from about 90-97 mol % and in particular at least about 95 mol % of vinylamine monomer units.

A certain molecular weight range of poly(vinylamine-vinylformamide) copolymers has also been shown to be particularly effective in reducing perspiration. Here, antiperspirant cosmetic products as contemplated herein are preferred, in which the poly(vinylamine-vinylformamide) copolymer has a weight average molecular weight of from about 500 to about 6000 g/mol, preferably of from about 1000 to about 3000 g/mol. The average molecular weight Mw can be determined for example by gel permeation chromatography (GPC) (Andrews P: “Estimation of the Molecular Weights of Proteins by Sephadex Gel-Filtration”; Biochem. J., 1964, 91, pages 222 to 233).

A particularly suitable commercial product that shows excellent results in the context of the present disclosure is Lupamin® 1595 from BASF, which has a degree of hydrolysis (vinylamine monomer content) of approx. 95% and a molecular weight of <10 kDa.

The poly(vinylamine-vinylformamide) copolymers used as contemplated herein are usually marketed in aqueous dispersion/solution. In the context of the present disclosure, it has been found to be preferable to adsorb these dispersions on solids and to use them in the form of active substance-loaded particles. The effectiveness is particularly increased if the polymer particles meet certain particle size criteria.

Here, particularly preferred antiperspirant cosmetics as contemplated herein include the poly(vinylamine-vinylformamide) copolymer adsorbed on solids, and the solids are used in the form of particles which fall about 100% through a sieve with a mesh size of about 200 μm, preferably which fall about 100% through a sieve with a mesh size of about 100 μm, whereby it is extremely preferred that in addition at least about 30% by weight of the particles remain on a sieve with a mesh size of about 10 μm.

Essential for the present disclosure is that the cosmetic product has a neutral to acidic pH value of from about 2.5 to about 7.5 (determined at about 22° C.). The pH value of the cosmetic agent as contemplated herein is preferably adjusted to a range of from about 2.5 to about 7.0, more preferably from about 3.0 to about 5.5, even more preferably from about 3.3 to about 4.5. As already mentioned, aqueous solutions of poly(vinylamine-vinylformamide) copolymers have an alkaline pH value. In order to bring the pH value into the range claimed in the present disclosure, a pH adjusting agent is used. As contemplated herein, inorganic acids such as hydrochloric acid, phosphoric acid and/or sulphuric acid are particularly suitable for this purpose. Accordingly, the cosmetic composition as contemplated herein contains chloride ions, sulphate ions and/or phosphate ions as inorganic anions, whereby chloride ions are particularly preferred. The best antiperspirant effect was observed with chloride ions. Preferred designs therefore contain chloride as an inorganic anion. Preferably the proportion of chloride ions is about 10%, about 30%, about 50%, about 95.0% or more by weight, more preferably from about 99.0% to about 100% by weight of all anions contained. As contemplated herein, sulphate ions also include hydrogen sulphates. As contemplated herein, phosphates also include hydrogen phosphates and dihydrogen phosphates, and polyphosphates.

Extremely preferred antiperspirant cosmetic compositions as contemplated herein include anions, where at least about 10, preferably at least about 30%, more preferably at least about 50%, even more preferably at least about 95.0% and in particular from about 99.0% to about 100% by weight of the anions contained are chloride ions.

In another embodiment of the present disclosure, in addition to chloride, sulphate and/or phosphate ions, at least one further anion may be contained. Further anions can be inorganic anions or organic anions. The organic anions can be for example phosphonates, citrate, lactate, tartrate and/or malate. Preferred antiperspirant cosmetic compositions as contemplated herein contain at least one further anion, preferably an organic anion, which is further preferably selected from the group of citrate, lactate, tartrate and malate and/or other anions of organic acids.

In extremely preferred embodiments of the present disclosure, no anions other than chloride ions, sulphate ions and/or phosphate ions are contained.

The antiperspirant effect is based on influencing the sweat gland(s). As contemplated herein, influencing the sweat gland(s) is understood to mean influencing the sweat gland(s) in such a way that the secretion of sweat from the excretory duct is avoided or reduced.

The cosmetic product as contemplated herein therefore contains at least one emulsifier. The cosmetic product of the present disclosure may be formulated as an oil-in-water emulsion or a water-in-oil emulsion. In the context of the present disclosure, it is preferred if the antiperspirant cosmetic is in the form of an oil-in-water emulsion. In this case, the cosmetic product as contemplated herein is preferably sprayed as a propellant-free pump spray or a squeeze spray or applied as roll-on. However, within the scope of the present disclosure, the antiperspirant cosmetic product may also be present as a water-in-oil emulsion. This may in particular be a sprayable water-in-oil emulsion which can be sprayed by employing a propellant.

The antiperspirant cosmetic agent of the present disclosure may be present as an aqueous, aqueous-alcoholic, aqueous-glycolic solution and/or as an emulsion. The cosmetic product can be a thin fluid or a product with adjusted viscosity (flow behaviour).

The at least one emulsifier is contained in a total amount of from about 0.05 to about 10.0% by weight, preferably from about 0.2 to about 5.0% by weight, more preferably from about 1.0 to about 4.0% by weight, even more preferably from about 1.5 to about 3.0% by weight.

Emulsifiers suitable as contemplated herein can be selected from anionic, cationic, non-ionic, amphoteric, in particular ampholytic and zwitterionic emulsifiers. However, since the poly(vinylamine-vinylformamide) copolymer is present together with certain anions, in particular chloride, non-ionic emulsifiers are particularly preferred. Therefore, the cosmetic product as contemplated herein contains only non-ionic emulsifiers in preferred forms.

### Oil-In-Water Emulsifiers

The cosmetic compositions as contemplated herein, which are formulated as an emulsion, in particular as an oil-in-water emulsion, preferably contain at least one non-ionic oil-in-water emulsifier with an HLB value of more than from about 7 to about 20. These are emulsifiers generally known to experts. For ethoxylated products, the HLB value is calculated according to the formula HLB=(100−L):5, where L is the percentage by weight of lipophilic groups, i.e. fatty alkyl or fatty acyl groups, in the ethylene oxide adducts, expressed in weight percent. When selecting non-ionic oil-in-water emulsifiers which are suitable as contemplated herein, it is particularly preferred to use a mixture of non-ionic oil-in-water emulsifiers in order to be able to optimally adjust the stability of the compositions as contemplated herein. The individual emulsifier components provide a proportion of the total HLB value or average HLB value of the oil-in-water emulsifier mixture according to their proportion of the total quantity of oil-in-water emulsifiers. As contemplated herein, the average HLB value of the oil-in-water emulsifier mixture is from about 10-19, preferably from about 12-18 and particularly preferably from about 14-17. In order to achieve such average HLB values, oil-in-water emulsifiers from the HLB value ranges from about 10-14, from about 14-16 and if necessary from about 16-19 are preferably combined with each other. Of course, the oil-in-water emulsifier mixtures can also contain non-ionic emulsifiers with HLB values in the range of >7-10 and from about 19-20; such emulsifier mixtures may also be preferred as contemplated herein. However, the deodorant or antiperspirant compositions of the present disclosure may also contain a single oil-in-water emulsifier with an HLB value in the range from about 10-19 in another preferred form. Preferred deodorant or antiperspirant compositions of the present disclosure contain at least one non-ionic oil-in-water emulsifier selected from ethoxylated C₈-C₂₄ alkanols with an average of about 10-100 moles of ethylene oxide per mole, ethoxylated C₈-C₂₄ carboxylic acids with an average of about 10-100 moles of ethylene oxide per mole, silicone copolyols with ethylene oxide units or with ethylene oxide and propylene oxide units, Alkyl mono- and oligoglycosides with about 8-22 carbon atoms in the alkyl residue and their ethoxylated analogues, ethoxylated sterols, partial esters of polyglycerols with from 2 to about 10 glycerol units and esterified with 1-4 saturated or unsaturated, linear or branched, optionally hydroxylated C₈-C₃₀ fatty acid residues, provided they have an HLB value of more than about 7, and mixtures of the aforementioned substances. The ethoxylated C₈-C₂₄ alkanols have the formula R¹O(CH₂CH₂O)ₙH, where R¹ is a linear or branched alkyl and/or alkenyl radical having about 8-24 carbon atoms and n, the average number of ethylene oxide units per molecule, are numbers from about 10-100, preferably about 10-30 moles of ethylene oxide to 1 mole of caprylic alcohol, 2-Ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and their technical mixtures. Adducts of about 10-100 mol ethylene oxide to technical fatty alcohols with about 12-18 carbon atoms, such as coconut, palm, palm kernel or tallow fatty alcohol, are also suitable.

Preferred cosmetic compositions contain a non-ionic emulsifier from the group of polyalkylene glycol ethers, preferably from the group of alkoxylated C₈-C₂₄ alkanols with an average of about 10-100 mol alkylene oxide per mole, preferably from the group of ethoxylated C₁₂-C₁₈ alkanols with an average of about 10-30 mol ethylene oxide per mole.

The ethoxylated C₈-C₂₄ carboxylic acids have the formula R¹(OCH₂CH₂)ₙOH, where R¹ is a linear or branched saturated or unsaturated acyl radical having about 8-24 carbon atoms and n, the average number of ethylene oxide units per molecule, are numbers from about 10-100, preferably about 10-30 moles of ethylene oxide to 1 mole of caprylic acid, 2-ethylhexanoic acid, capric acid, lauric acid, isotridecanoic acid, myristic acid, cetyl acid, palmitoleic acid, stearic acid, isostearic acid, oleic acid, elaidic acid, petroselinic acid, arachidic acid, gadoleic acid, behenic acid, erucic acid and brassidic acid as well as their technical mixtures. Adducts of about 10-100 mol ethylene oxide to technical fatty acids with about 12-18 carbon atoms, such as coconut, palm, palm kernel or tallow fatty acid, are also suitable. Especially preferred are PEG-50-monostearate, PEG-100-monostearate, PEG-50-monooleate, PEG-100-monooleate, PEG-50-monolaurate and PEG-100-monolaurate. The C12-C18 alkanols or the C12-C18 carboxylic acids with about 10-30 units of ethylene oxide per molecule as well as mixtures of these substances, especially Ceteth-12, Ceteth-20, Ceteth-30, Steareth-12, Steareth-20, Steareth-30, Laureth-12 and Beheneth-20 are particularly preferred. Furthermore, C8-C22 alkyl mono- and oligoglycosides are preferred. C₈-C₂₂ alkyl mono- and oligoglycosides are well-known commercial surfactants and emulsifiers. They are produced in particular by reacting glucose or oligosaccharides with primary alcohols with 8-22 carbon atoms. With regard to the glycoside residue, both monoglycosides, in which a cyclic sugar residue is glycosidically bound to the fatty alcohol, and oligomeric glycosides with a degree of oligomerization up to about 8, preferably 1-2, are suitable. The degree of oligomerization is a statistical mean value based on a homologue distribution that is common for such technical products. Products available under the name Plantacare® contain a glucosidically bound C₈-C₁₆ alkyl group on an oligoglucoside residue whose average degree of oligomerization is 1-2, in particular about 1.1-1.4. Particularly preferred C₈-C₂₂ alkyl mono- and oligoglycosides are selected from octylglucoside, decylglucoside, laurylglucoside, palmitylglucoside, isostearylglucoside, stearylglucoside, arachidylglucoside and behenylglucoside and mixtures thereof. The acylglucamides derived from glucamine are also suitable as non-ionic oil-in-water emulsifiers. Ethoxylated sterols, especially ethoxylated soyasterols, are also suitable oil-in-water emulsifiers. The degree of ethoxylation must be greater than about 5, preferably at least about 10, to have an HLB value greater than from about 7 to about 20. Suitable commercial products are for example PEG-10 Soy Sterol, PEG-16 Soy Sterol and PEG-25 Soy Sterol. Furthermore, partial esters of polyglycerols with 2 to 10 glycerol units and esterified with from 1 to 4 saturated or unsaturated, linear or branched, optionally hydroxylated C8-C30 fatty acid residues are preferably used, provided they have an HLB value in the range from more than from about 7 to about 20. Particularly preferred are diglycerinmonocaprlate, diglycerine monocaprate, diglycerine monolaurate, triglycerine monocaprylate, triglycerine monocaprate, triglycerine monolaurate, tetraglycerol monocaprylate, tetraglycerine monocaprate, tetraglycerol monocapatte, pentaglycerin monocaprylate, pentaglycerin monocaprate, pentaglycerin monolaurate, hexaglycerin monocaprylate, hexaglycerin monocaprate, hexaglycerin monolaurate, hexaglycerol monolaurate, hexaglycerin monocrate, hexaglycerol monocrate, hexaglycerin monocrate, decaglycerinmonocaprylate, decaglycerin monocaprate, decaglycerinmonolaurate, decaglycerinmonori state, decaglycerin monoisostarate, decaglycerin monostearate, decaglycerin monooleate, decaglycerinmono hydroxystearate, decaglycerin dicaprylate, decaglycerin dicaprate, decaglycerin diedilaurate, decaglycerin dimyristate, decaglycerin diisosstearate, decaglycerin distearrate, decaglycerin diolelate, decaglycerindihydroxysteate, decaglycerintricaprylate, decaglycerintricaprate, decaglycerintritrilaurate, decaglycerintrimyristate, decaglycerintriisostestearate, decaglycerintristerate, decaglycerintrioleate and decaglycerol trihydroxystearate. Other particularly suitable oil-in-water emulsifiers are polyethylene glycol derivatives of hydrogenated castor oil (PEG-Hydrogenated Castor Oil). Particularly preferred deodorant or antiperspirant compositions as contemplated herein include a non-ionic oil-in-water emulsifier in a total amount of from about 0.5-10% by weight, particularly preferably from about 1-4% by weight and extremely preferably from about 1.5-3% by weight, based on the total composition.

### Water-In-Oil Emulsifiers

Cosmetic compositions preferred as contemplated herein preferably further contain at least one non-ionic water-in-oil emulsifier with an HLB value greater than about 1.0 and less than or equal to about 7.0, selected from the mono- and diesters of ethylene glycol and the mono-, di-, tri- and tetraesters of pentaerythritol with linear saturated fatty acids with about 12-30, in particular about 14-22 carbon atoms, which may be hydroxylated, as well as mixtures thereof, as a consistency enhancer and/or a water binder. As contemplated herein, the mono-and diesters are preferred. C₁₂-C₃₀ fatty acid residues preferred by the present disclosure are selected from lauric acid, myristic acid, palmitic acid, stearic acid, arachidic acid and behenic acid residues; the stearic acid residue is particularly preferred. Non-ionic water-in-oil emulsifiers having an HLB value greater than about 1.0 and less than or equal to about 7.0 are selected from pentaerythrityl monostearate, pentaerythrityl distearate, pentaerythrityl tristearate, pentaerythrityl tetrastearate, ethylene glycol monostearate, ethylene glycol distearate and mixtures thereof. As contemplated herein, water-in-oil emulsifiers with an HLB value greater than about 1.0 and less than or equal to about 7.0 are particularly preferred as commercial products, for example Cutina® PES (INCI: Pentaerythrityl distearate), Cutina® AGS (INCI: Glycol distearate) or Cutina® EGMS (INCI: Glycol stearate) are available. These commercial products already represent mixtures of mono- and diesters (the pentaerythrityl esters also contain tri- and tetraesters). As contemplated herein, it may be preferred to use only a single water-in-oil emulsifier. In another preferred form, the compositions as contemplated herein contain mixtures, in particular technical mixtures, of at least two water-in-oil emulsifiers. A technical mixture is understood to be a commercial product such as Cutina® PES. In addition to the above-mentioned water-in-oil emulsifiers based on ethylene glycol or pentaerythrityl esters, at least one further non-ionic water-in-oil emulsifier with an HLB value greater than about 1.0 and less than or equal to about 7.0 may also be present in a preferred form, but its proportion of the total weight of non-ionic water-in-oil emulsifiers with an HLB value greater than about 1.0 and less than or equal to about 7.0 should preferably not be greater than 80%. In a particularly preferred form, the compositions as contemplated herein contain at least one additional water-in-oil emulsifier with an HLB value greater than about 1.0 and less than or equal to about 7.0 only in a proportion by weight of not more than about 5% or are free from additional water-in-oil emulsifiers. For ethoxylated adducts, the HLB value can also be calculated, as mentioned above. Preferred water-in-oil emulsifiers are:
linear saturated alkanols with about 12-30 carbon atoms, especially with about 16-22 carbon atoms, especially cetyl alcohol, stearyl alcohol, arachidyl alcohol, behenyl alcohol and lanolin alcohol or mixtures of these alcohols, as obtainable in the technical hydrogenation of vegetable and animal fatty acids,
Esters and especially partial esters of a polyol with 3-6 C atoms and linear saturated and unsaturated fatty acids with about 12-30, especially about 14-22 C atoms, which may be branched and/or hydroxylated. Such esters or partial esters are for example the mono- and diesters of glycerol or the monoesters of propylene glycol with linear or branched, saturated and unsaturated C-C carboxylic acids which may be hydroxylated, in particular those with palmitic, isostearic and stearic acids, the sorbitan mono-, di- or triesters of linear saturated and unsaturated C-C carboxylic acids, which may be hydroxylated, in particular those of myristic acid, palmitic acid, stearic acid or mixtures of these fatty acids and the methylglucose mono-and diesters of linear saturated and unsaturated C—C carboxylic acids which may be hydroxylated;
Sterols, i.e. steroids which carry a hydroxyl group on the C3 atom of the steroid skeleton and are isolated from animal tissue (zoosterols, e.g. cholesterol, lanosterol) as well as from plants (phytosterols, e.g. ergosterol, stigmasterol, sitosterol) and from fungi and yeasts (mycosterols) and which can be low ethoxylated (1-5 EO);
Alkanols and carboxylic acids each having about 8-24 C atoms, in particular about 16-22 C atoms, in the alkyl group and 1-4 ethylene oxide units per molecule, which have an HLB value greater than about 1.0 and less than or equal to about 7.0,
Glycerol monoethers of saturated and/or unsaturated, branched and/or unbranched alcohols having a chain length of about 8-30, in particular about 12-18 carbon atoms
Partial esters of polyglycerols with n=from 2 to about 10 glycerol units and esterified with from 1 to 5 saturated or unsaturated, linear or branched, optionally hydroxylated C—C fatty acid residues, provided they have an HLB value of about 7 or less,
and mixtures of the aforementioned substances.

As contemplated herein, it may be preferred to use only one additional water-in-oil emulsifier. In another preferred form, the compositions of the present disclosure contain mixtures, in particular technical mixtures, of at least two additional water-in-oil emulsifiers. A technical mixture is, for example, a commercial product such as Cutina® GMS, which is a mixture of glyceryl monostearate and glyceryl distearate. Additional water-in-oil emulsifiers that can be used to particular advantage are stearyl alcohol, cetyl alcohol, glyceryl monostearate, especially in the form of the commercial products Cutina® GMS and Cutina® MD (ex Cognis), glyceryl distearate, glyceryl monocaprinate, glyceryl monocaprylate, glyceryl monolaurate, glyceryl monomyristate, glyceryl monopalmitate, glyceryl monohydroxystearate, glyceryl monooleate, glyceryl monolanolate, glyceryl dimyristate, glyceryl dipalmitate, glyceryl diolate, propylene glycol monostearate, propylene glycol monolaurate, sorbitan monocaprylate, sorbitan monolaurate, sorbitan monomyristate, sorbitan monopalmitate, sorbitan monostearate, sorbitan sesquistearate, sorbitan distearate, sorbitan dioleate, sorbitan sesquioleate, sucrose distearate, arachidyl alcohol, behenyl alcohol, polyethylene glycol(2) stearyl ether (steareth-2), steareth-5, oleth-2, diglycerol monostearate, diglycerol monoisostearate, diglycerol monooleate, diglycerol dihydroxystearate, diglycerol distearate, diglycerol dioleate, triglycerol distearate, tetraglycerol monostearate, tetraglycerol distearate, tetraglycerol tristearate, decaglycerol pentastearate, decaglycerol pentahydroxystearate, decaglycerol pentaisostearate, decaglycerol pentaoleate, soy sterol, PEG-1 soy sterol, PEG-5 soy sterol, PEG-2-monolaurate and PEG-2-monostearate.

Preferred deodorant or antiperspirant compositions as contemplated herein include at least one water-in-oil emulsifier in a total amount of from about 0.1-10% by weight, preferably from about 0.5-8.0% by weight, and particularly preferably from about 1-4% by weight, based on the total composition. Furthermore, quantities of from about 2-3% by weight, based on the total weight of the composition, may also be extremely preferred as contemplated herein. The HLB values can be calculated according to Griffin. If there are different indications of the HLB value of a substance in the literature, the HLB value that comes closest to the value calculated according to Griffin should be used for teaching as contemplated herein. If no clear HLB value can be determined in this way, the HLB value specified by the manufacturer of the emulsifier is to be used for teaching as contemplated herein. If this is also not possible, the HLB value should be determined experimentally.

A group of water-in-oil emulsifiers which is, as contemplated herein, particularly preferred are the poly-(C₂-C₃)alkylene glycol-modified silicones, whose former INCI designation was dimethicone copolyol, with the current INCI designations PEG-x Dimethicone (with x=from about 2-20, preferably from about 3-17, particularly preferably from about 11-12), Bis-PEG-y Dimethicone (with y=from about 3-25, preferably from about 4-20), PEG/PPG a/b Dimethicone (where a and b are independent of each other represent numbers from about 2-30, preferably from about 3-30 and more preferably from about 12-20, in particular from about 14-18), Bis-PEG/PPG-c/d dimethicones (where c and d independently represent numbers from about 10-25, preferably from about 14-20 and more preferably from about 14-16) and Bis-PEG/PPG-e/f PEG/PPG g/h dimethicones (where e, f, g and h independently represent numbers from about 10-20, preferably from about 14-18 and more preferably about 16). Particularly preferred are PEG/PPG-18/18 dimethicone, which is produced in a 1:9 mixture with cyclomethicone as DC 3225 C or DC 5225 C, or as a mixture with dimethicone as DC 5227 DM, PEG/PPG-4/12 dimethicone, which is available in the trade under the name Abil® B 8852, and Bis-PEG/PPG-14/14 dimethicone, which is commercially available in a mixture with cyclomethicone as Abil® EM 97 (Goldschmidt), Bis-PEG/PPG-20/20 dimethicone, which is commercially available as Abil® B 8832, PEG/PPG-5/3 trisiloxanes (Silsoft® 305), and PEG/PPG-20/23 dimethicone (Silsoft® 430 and Silsoft® 440). Other W/O emulsifiers preferred by the present disclosure are poly-(C2-C3)alkylene glycol-modified silicones which are hydrophobically modified with C4-C18 alkyl groups, particularly preferably cetyl PEG/PPG-10/1 dimethicones (formerly: cetyl dimethicone copolyol, obtainable as Abil® EM 90 or in a mixture of polyglyceryl 4-isostearate, cetyl PEG/PPG-10/1 dimethicone and hexyl laurate under the trade name Abil® WE 09), furthermore alkyl methicone copolyols.

The cosmetic compositions of the present disclosure preferably further contain at least one oil component in a total amount of from about 0.05 to about 15% by weight. As contemplated herein, the term oil component refers to cosmetic oils. For the purposes of the present disclosure, the term “cosmetic oil” means an oil suitable for cosmetic use which is not miscible with water in all quantities. The cosmetic oil used as contemplated herein is neither a fragrance nor an essential oil.

The cosmetic compositions as contemplated herein contain as cosmetic oil at least one substance selected from the group of cosmetic oils which are liquid at about 20° C. and about 1013 hPa.

In the context of the present disclosure, the cosmetic oil liquid at about 20° C. and about 1013 hPa is selected from the group of (i) volatile silicone oils, in particular cyclic and linear silicone oils; (ii) volatile non-silicone oils, in particular liquid paraffin oils and isoparaffin oils; (iii) non-volatile silicone oils; (iv) non-volatile non-silicone oils; and (v) mixtures thereof.

As contemplated herein, the term “volatile oil” refers to oils which, at about 20° C. and an ambient pressure of about 1,013 hPa, have a vapour pressure of from about 2.66 Pa to about 40,000 Pa (from about 0.02 to about 300 mm Hg), preferably of from about 10 to about 12,000 Pa (from about 0.1 to about 90 mm Hg), more preferably of from about 13 to about 3,000 Pa (from about 0.1 to about 23 mm Hg), in particular of from about 15 to about 500 Pa (from about 0.1 to about 4 mm Hg).

In addition, for the purposes of the present disclosure, the term “non-volatile oils” means oils having a vapour pressure of less than about 2.66 Pa (about 0.02 mm Hg) at about 20° C. and an ambient pressure of about 1,013 hPa.

As contemplated herein, it may be preferable to use mixtures of volatile silicone oils and volatile non-silicone oils in the antiperspirant cosmetic products, as a drier skin feeling is achieved. Furthermore, within the scope of the present disclosure, it may be preferred if the antiperspirant cosmetic products contain a non-volatile silicone oil and/or a non-volatile non-silicone oil in order to mask insoluble components such as talcum or ingredients dried on the skin.

As contemplated herein, the use of blends of non-volatile and volatile cosmetic oils is particularly preferred, since in this way parameters such as skin feel, visibility of the residue and stability of the antiperspirant cosmetic product as contemplated herein can be adjusted and the product can thus be better adapted to the needs of consumers.

Volatile cosmetic oils are usually selected from cyclic silicone oils with the INCI designation cyclomethicone. The INCI designation Cyclomethicone means in particular cyclotrisiloxane (hexamethylcyclotrisiloxane), cyclotetrasiloxane (octamethylcyclotetrasiloxane), cyclopentasiloxane (decamethylcyclopentasiloxane) and cyclohexasiloxane (dodecamethylcyclohexasiloxane). These oils have a vapour pressure of from about 13-15 Pa at about 20° C. Cyclomethicones are known in the state of the art as well-suited oils for cosmetic compositions, in particular for deodorant compositions such as sprays and pens. However, due to their persistence in the environment, it may be preferable, as contemplated herein, not to use cyclomethicones. In a particularly preferred embodiment, the compositions as contemplated herein and used as contemplated herein contain 0 to less than about 1% by weight, preferably at most about 0.1% by weight, of cyclomethicones, based on the weight of the composition, not taking into account any blowing agent present. A cyclomethicone substitute preferred as contemplated herein is a mixture of C₁₃-C₁₆ isoparaffins, C₁₂-C₁₄ isoparaffins and C₁₀-C₁₅ alkanes, whose viscosity at about 25° C. is in the range from about 2 to about 6 mPas and which has a vapour pressure at about 20° C. in the range from about 10 to about 150 Pa, preferably from about 100 to about 150 Pa. Such a mixture is for example available under the name SiClone® SR-5 from the company Presperse Inc. Other preferred volatile silicone oils are selected from volatile linear silicone oils, especially volatile linear silicone oils with from about 2-10 siloxane units, such as hexamethyldisiloxane (L2), octamethyltrisiloxane (L3), decamethyltetrasiloxane (L4), such as are contained in the commercial products DC 2-1184, Dow Corning® 200 (0.65 cSt) and Dow Corning® 200 (1.5 cSt) from Dow Corning®, and low molecular weight phenyl trimethicone with a vapor pressure at 20° C. of about 2000 Pa, such as is available from GE® Bayer Silicones/Momentive under the name Baysilone Fluid PD 5. Preferred antiperspirant compositions as contemplated herein contain at least one volatile silicone oil, which may be cyclic or linear, due to the drier skin feel and faster release of the active ingredient. Other preferred products as contemplated herein contain at least one volatile non-silicone oil due to the drier skin feel and the faster release of the active agent. Preferred non-silicone volatile oils are selected from C₈-C₁₆ isoparaffins, especially isonononane, isodecane, isoundecane, isododecane, isotridecane, isotetradecane, isopentadecane and isohexadecane, and mixtures thereof. Preferred are C₁₀-C₁₃ isoparaffin mixtures and/or C₁₀-C₁₈ n-paraffin mixtures, especially those with a vapour pressure at about 20° C. of from about 10-400 Pa, preferably from about 13-100 Pa. Other agents preferred by the present disclosure contain at least one non-volatile cosmetic oil selected from non-volatile silicone oils and non-volatile non-silicone oils. Residues of components that are insoluble in the composition, such as antiperspirant agents (=antiperspirant aluminium salts) or talcum, can be successfully masked with a non-volatile oil. In addition, a mixture of different oils, in particular non-volatile and volatile oils, can be used to fine-tune parameters such as skin feel, the visibility of the residue and the stability of the composition as contemplated herein and to better adapt it to the needs of consumers. The cosmetic oil, which is neither a fragrance nor an essential oil, comprises at least one volatile oil with a vapour pressure of from about 10-3000 Pa at about 20° C., which is neither a fragrance nor an essential oil, in a total amount of from about 0.1-100% by weight, particularly preferably from about 10-90% by weight, each based on the total weight of the cosmetic oils.

Of course, it is also possible to formulate agents as contemplated herein with a low percentage of volatile oils relative to the total weight of the agent or even without volatile oils. As contemplated herein, particularly preferred oils are esters of linear or branched saturated or unsaturated fatty alcohols with about 2-30 carbon atoms with linear or branched saturated or unsaturated fatty acids with about 2-30 carbon atoms, which may be hydroxylated. It should be noted that some esters of linear or branched C₁-C₂₂ alkanols or C₁₄-C₂₂ alkenols and some triesters of glycerol with linear or branched C₂-C₂₂ carboxylic acids, which may be saturated or unsaturated, are solid under normal conditions, such as cetylstearate or glycerol tristearate (=stearin). As contemplated herein, these esters, which are solid under normal conditions, do not constitute cosmetic oils since they do not meet the condition “liquid under normal conditions”. The classification whether such an ester is liquid or solid under normal conditions is within the scope of the expert's general knowledge. Preferred are esters of linear or branched saturated fatty alcohols with about 2-18 carbon atoms with linear or branched saturated or unsaturated fatty acids with about 3-18 carbon atoms, which may be hydroxylated. Preferred examples are isopropyl palmitate, isopropyl stearate, isopropyl myristate, 2-hexyl decyl stearate, 2-hexyl decyl laurate, isodecyl neopentanoate, isononylisononanoate, 2-ethylhexyl palmitate and 2-ethylhexyl stearate. Also preferred are isopropyl isostearate, isopropyl oleate, isooctyl stearate, is ononyl stearate, isocetylstearate, isononylisononanoate, isotridecylisononanoate, Cetearylisononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate, n-hexyl laurate, n-decyl oleate, oley oleate, oleylerucate, erucyl oleate, erucylerucate, ethylene glycol dioleate, ethylene glycol dipalmitate, n-hexyl laurate, n-decyl oleate, oleyl oleate, oleylerucate, erucyl oleate, C12-C15 alkyl lactate and di-C12-C13 alkyl malate and the benzoic acid esters of linear or branched C8-22 alkanols. Benzoic acid C₁₂-C₁₅ alkyl esters are particularly preferred, e.g. available as commercial product. Other oil components preferred as contemplated herein are selected from the C₈-C₂₂ fatty alcohol esters of monovalent or polyvalent C₂-C₇ hydroxycarboxylic acids, in particular the esters of glycolic acid, lactic acid, malic acid, tartaric acid, citric acid and salicylic acid. Such esters based on linear C14/15 alkanols, e.g. C12-C15 alkyl lactate, and on C12/13 alkanols branched in the 2-position are available under the trade name Cosmacol® from the company Nordmann®, Rassmann GmbH & Co, Hamburg, in particular the trade products Cosmacol® ESI, Cosmacol® EMI and Cosmacol® ETI, Finsolv® TN (C12-C15 alkyl benzoate), as well as benzoic acid ostearyl esters, e.g. available as Finsolv® SB, 2-ethylhexyl benzoate, e.g. available as Finsolv® EB, and 2-octyldodecyl benzoate, e.g. available as Finsolv® BOD.

Another particularly preferred ester oil is triethyl citrate.

Preferred cosmetic compositions contain an ester of a C3-C30 carboxylic acid, preferably an ester from the group 2-ethylhexyl palmitate, isopropyl palmitate and triethyl citrate.

Products favoured by the present disclosure may contain branched saturated or unsaturated fatty alcohols with about 6-30 carbon atoms. These are often referred to as Guerbet alcohols because they are available after the Guerbet reaction. Preferred alcohol oils are 2-hexyldecanol, 2-octyldodecanol and 2-ethylhexyl alcohol. Isostearyl alcohol is also preferred. Other preferred non-volatile oils are selected from mixtures of Guerbet alcohols and Guerbet alcohol esters, e.g. 2-hexyl decanol and 2-hexyl decyl laurate. The term ‘triglyceride’ used in the following refers to ‘glycerol triesters’. Other non-volatile oils preferred by the present disclosure are selected from the triglycerides of linear or branched, saturated or unsaturated, optionally hydroxylated C8-30 fatty acids, provided they are liquid under normal conditions. The use of natural oils, e.g. soy bean oil, cottonseed oil, sunflower oil, palm oil, palm kernel oil, linseed oil, almond oil, castor oil, corn oil, rapeseed oil, olive oil, sesame oil, thistle oil, wheat germ oil, peach kernel oil and the liquid portions of coconut oil and the like may be particularly suitable. Synthetic triglyceride oils, especially capric/caprylic triglycerides, e.g. the commercial products Myritol® 318 or Myritol® 331 (BASF®/Cognis) with unbranched fatty acid residues and glyceryl triisostearin and glyceryl tri(2-ethylhexanoate) with branched fatty acid residues, are particularly preferred. Such triglyceride oils preferably account for less than about 50% by weight of the total weight of all cosmetic oils in the composition as contemplated herein. In particular, the total weight of triglyceride oils is from about 0.5-10% by weight, preferably from about 1-5% by weight, in each case based on the total composition, not considering any blowing agent present. Further non-volatile non-silicone oils which are particularly preferred as contemplated herein are selected from the dicarboxylic acid esters of linear or branched C2-C10 alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate. Other non-volatile non-silicone oils which are particularly preferred as contemplated herein are selected from the addition products of 1 to 5 propylene oxide units to mono- or polyvalent C8-22 alkanols such as octanol, decanol, decanediol, lauryl alcohol, myristyl alcohol and stearyl alcohol, e.g. PPG-2-myristyl ether and PPG-3-myristyl ether. Further non-volatile non-silicone oils which are particularly preferred as contemplated herein are selected from the addition products of at least 6 ethylene oxide and/or propylene oxide units to mono- or polyvalent C₃-C₂₂ alkanols such as glycerol, butanol, butanediol, myristyl alcohol and stearyl alcohol, which, if desired, can be esterified, e.g. PPG-14 butyl ether, PPG-9 butyl ether, PPG-10 butanediol and PPG-15 stearyl ether. Other non-volatile non-silicone oils that are particularly preferred as contemplated herein are selected from the symmetrical, asymmetrical or cyclic esters of carbonic acid with C6-C20 alcohols, e.g. di-n-caprylylcarbonate (Cetiol® CC) or di-(2-ethylhexyl) carbonate (Tegosoft® DEC). However, esters of carbonic acid with C₁-C₅ -alcohols, e.g. glycerin carbonate or propylene carbonate, are not suitable as cosmetic oils. Other oils which may be preferred as contemplated herein are selected from the esters of dimers of unsaturated C₁₂-C₁₈ fatty acids (dimer fatty acids) with monovalent linear, branched or cyclic C₂-C₁₈ alkanols or with polyvalent linear or branched C₂-C₆ alkanols. In particular, the total weight of dimer fatty acid esters is from about 0.1-10% by weight, preferably from about 1-5% by weight, in each case based on the total composition, not considering any blowing agent present. Other cosmetic oils which are particularly preferred as contemplated herein are selected from non-volatile silicone oils. Non-volatile silicone oils preferred as contemplated herein are selected from linear polyalkylsiloxanes with a kinematic viscosity at about 25° C. of at least from about 5 cSt to about 2000 cSt, in particular selected from linear polydimethylsiloxanes with a kinematic viscosity at about 25° C. of from about 5 cSt to about 2000 cSt, preferably from about 10 to about 350 cSt, particularly preferably from about 50-100 cSt, as they are obtainable, for example, under the trade names Dow Corning® 200 or Xiameter® PMX from Dow Corning® or Xiameter®. Other preferred non-volatile silicone oils are phenyltrimethicones with a kinematic viscosity at about 25° C. of from about 10 to about 100 cSt, preferably from about 15-30 cSt, and cetyldimethicones. Natural and synthetic hydrocarbons preferred as contemplated herein are selected from paraffin oils, isohexadecane, isoeicosane, polyisobutenes and polydecenes, which are available e.g. under the designation Emery® 3004, 3006, 3010 or under the designation Ethylflo® from Albemarle or Nexbase® 2004G from Nestle®, as well as 1,3-di(2-ethylhexyl)-cyclohexane. Especially preferred mixtures are free of volatile silicones or alkanes (linear/branched).

The cosmetic products as contemplated herein may still contain at least one perfume. For the purposes of the present disclosure, the term “perfumes” means substances with a molecular weight of from about 74 to about 300 g/mol which contain at least one osmophoric group in the molecule and have an odor and/or taste, i.e. they are able to excite the receptors of the hair cells of the olfactory system. Osmophoric groups are groups covalently bound to the molecular skeleton in the form of hydroxy groups, formyl groups, oxo groups, alkoxycarbonyl groups, nitrile groups, nitro groups, azide groups, etc. In this context, the term “perfumes” in the sense of the present disclosure also includes perfume oils, perfumes or perfume oil components which are liquid at about 20° C. and about 1013 hPa.

Preferably, however, mixtures of different fragrances are used, which together create an appealing scent.

Particularly appealing smelling antiperspirant cosmetic compositions as contemplated herein are obtained when the at least one fragrant substance is contained in a total amount of from about 0.001 to about 10% by weight, preferably from about 0.05 to about 9% by weight, preferably from about 0.01 to about 8% by weight, more preferably from about 0.1 to about 7% by weight, still more preferably from about 0.2 to about 6% by weight, in particular from about 0.2 to about 2% by weight, based on the total weight of the antiperspirant cosmetic composition.

Perfume oils may be present as free oil and/or in encapsulated form.

Furthermore, the antiperspirant cosmetic products as contemplated herein may contain a wax. In the context of the present disclosure, the term “wax” refers to substances which are kneadable or solid to brittle hard at about 20° C., have a coarse to fine crystalline structure and are translucent to opaque in colour, but not glassy. Furthermore, these substances melt above about 25° C. without decomposition, are slightly liquid (low viscosity) just above the melting point, have a strongly temperature-dependent consistency and solubility and can be polished under slight pressure.

Preferably this wax is selected from the group of (i) fatty acid glycerol mono-, di-and triesters; (ii) butyrospermum parkii (shea butter); (iii) esters of saturated C₈-C₁₈ monohydric alcohols with saturated C₁₂-C₁₈ monocarboxylic acids; (iv) linear primary C₁₂-C₂₄ alkanols; (v) esters of a saturated C₁₆-C₆₀ monohydric alkanol and a saturated C₈-C₃₆ monocarboxylic acid; (vi) glycerol triesters of saturated linear C₁₂-C₃₀ carboxylic acids which may be hydroxylated; (vii) natural vegetable waxes; (viii) animal waxes; (ix) synthetic waxes; and (x) mixtures thereof. In the context of the present disclosure preferably applicable waxes are disclosed in the disclosure document DE 10 2012 222 692 A1.

In the context of the present disclosure, it is preferred if the wax is contained in a total amount of from about 0.01 to about 30% by weight, in particular from about 6 to about 25% by weight, based on the total weight of the antiperspirant cosmetic.

According to one embodiment of the present disclosure, it may be provided that the antiperspirant cosmetic compositions as contemplated herein contain a propellant in a total amount of from about 0.1 to about 99% by weight, based on the total weight of the antiperspirant cosmetic composition. If the cosmetic compositions as contemplated herein contain a propellant, this is preferably contained in a total amount of from about 1 to about 98% by weight, preferably of from about 20 to about 90% by weight, preferably of from about 30 to about 85% by weight, in particular of from about 40 to about 75% by weight, based on the total weight of the antiperspirant cosmetic composition. In this case the cosmetic products as contemplated herein are packaged as propellant-driven aerosols. Preferred blowing agents (propellants) are propane, propene, n-butane, iso-butane, iso-butene, n-pentane, pentene, iso-pentane, iso-pentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropenes, both individually and in mixtures. Hydrophilic propellants, such as carbon dioxide, can also be used advantageously in the sense of the present disclosure if the proportion of hydrophilic gases is chosen to be low and lipophilic propellant (e.g. propane/butane) is present in excess. Propane, n-butane, iso-butane and mixtures of these propellants are particularly preferred. It has been shown that the use of n-butane as the only propellant gas can be particularly preferred as contemplated herein.

As contemplated herein it is still preferred if the antiperspirant cosmetic product additionally contains at least one preservative. As contemplated herein, the preferred preservatives are formaldehyde releasing iodopropynyl butylcarbamates, parabens, phenoxyethanol, ethanol, benzoic acid and its salts, dibromodicyanobutane, 2-bromo-2-nitro-propan-1,3-diol, imidazolidinyl urea, 5-chloro-2-methyl-4-isothiazolin-3-one, 2-chloroacetamide, benzalkonium chloride, benzyl alcohol, salicylic acid and salicylates. Further preservatives which may be used within the scope of the present disclosure are the substances listed in Annex 6 of the Cosmetics Ordinance as well as cosmetic raw materials with preservative properties or raw materials which support or enhance the preservative effect of the aforementioned preservatives. The preservatives are preferably contained in a total amount of from about 0.01 to about 10% by weight, preferably from about 0.1 to about 7% by weight, preferably from about 0.2 to about 5% by weight, in particular from about 0.3 to about 2.0% by weight, based on the total weight of the antiperspirant cosmetic.

As contemplated herein, the antiperspirant cosmetic composition may preferably continue to contain at least one antioxidant, preferably in a total amount of from about 0.01 to about 5.0 wt. %, based on the total weight of the cosmetic composition. The antioxidant may be selected from ascorbic acid, ascorbyl palmitate, BHT, substituted hydroxyhydrocinnamic acid esters, substituted benzotriazoles, dialkylthiodialkanoates, tocopherol and its esters (e.g. acetate), hydroxymethoxyphenyl decanones, paradol and mixtures thereof, in particular substituted hydroxyhydrocinnamic acid esters and mixtures thereof.

As contemplated herein, the antiperspirant cosmetic contains an aqueous cosmetic carrier. The antiperspirant cosmetic product contains free water preferably in an amount of from about 5 to about 99% by weight, based on the total weight of the antiperspirant cosmetic product. More preferably, the antiperspirant cosmetic contains free water in a total amount of from about 5 to about 96% by weight, preferably from about 15 to about 80% by weight, preferably from about 30 to about 70% by weight, in particular from about 40 to about 60% by weight, based on the total weight of the antiperspirant cosmetic. The aqueous cosmetic carrier may also contain ethanol in embodiments of the present disclosure. For example, the cosmetic composition of the present disclosure may contain from about 0.1 to about 70% by weight or from about 0.5 to about 30% or from about 1 to about 10% by weight of ethanol, based on the total weight of the antiperspirant cosmetic composition.

The application of the antiperspirant cosmetic product as contemplated herein can be carried out by various methods. According to a preferred design, the antiperspirant cosmetic product is packaged as a spray application. The spray application is carried out with a spraying device which contains a filling of the present disclosure's aqueous antiperspirant cosmetic agent in a container. The filling can be under the pressure of a propellant (compressed gas cans, compressed gas packages, aerosol packages), or it can be a mechanically operated pump sprayer without propellant (pump sprays/squeeze bottle). The antiperspirant cosmetic can be atomized physically, mechanically or electromechanically, for example by piezo effects or electric pumps.

The antiperspirant cosmetic product can also preferably be packaged as a pencil, soft solid, cream, gel or roll-on. The formulation of the antiperspirant cosmetic products as contemplated herein in a specific pharmaceutical form, such as an antiperspirant roll-on, an antiperspirant stick or an antiperspirant gel, is preferably based on the requirements of the intended use. Depending on the intended use, the antiperspirant cosmetic products as contemplated herein may therefore be in solid, semi-solid, liquid, disperse, emulsified, suspended, gel or multi-phase form. For the purposes of the present disclosure, the term liquid also includes any kind of solid dispersions in liquids. Furthermore, multi-phase antiperspirant cosmetic products according to the present disclosure are products which have at least 2 different phases with a phase separation and in which the phases can be arranged horizontally, i.e. one above the other, or vertically, i.e. next to each other. The application can be done for example with a roller ball applicator or by employing a fixed pin.

It may also be preferred in the context of the present disclosure if the antiperspirant cosmetic is contained on and/or in a disposable substrate selected from the group of cloths, pads and puffs. Particularly preferred are wet wipes, i.e. wet wipes which are prefabricated for the user, preferably individually packaged, as they are well known, for example, from the field of glass cleaning or wet toilet paper. Such wet wipes, which may advantageously also contain preservatives, are impregnated or coated with an antiperspirant cosmetic agent as contemplated herein and preferably individually packaged. Preferred substrate materials are selected from porous flat cloths. These cloths include cloths of woven and non-woven (non-woven) synthetic and natural fibres, felt, paper or foam, such as hydrophilic polyurethane foam. Preferred deodorizing or antiperspirant substrates as contemplated herein can be obtained by soaking or impregnation or also by melting an antiperspirant cosmetic agent onto a substrate.

As contemplated herein, the antiperspirant cosmetic composition preferably contains at least one further auxiliary substance selected from the group of thickening agents; chelating agents; deodorant agents; mono- and/or polyhydric alcohols and/or polyethylene glycols; skin-cooling agents; pH-adjusting agents; skin-care agents, such as moisturisers, skin-soothing agents, skin-lightening agents, skin-smoothing agents; and mixtures thereof. Particularly preferred antiperspirant cosmetic products as contemplated herein contain at least one thickening agent.

For thickening the antiperspirant cosmetic products as contemplated herein, substances are preferably used which are selected from cellulose ethers, xanthan gum, sclerotium gum, succinoglucans, polygalactomannans, pectins, agar, carrageenan, tragacanth, gum arabic, karaya gum, tara gum, gellan gum, gelatine, propylene glycol alginate, alginic acids and their salts, polyvinylpyrrolidones, polyvinyl alcohols, polyacrylamides, physical (e.g. physically (e.g. by pregelatinization) and/or chemically modified starches, acrylic acid-acrylate copolymers, acrylic acid-acrylamide copolymers, acrylic acid-vinylpyrrolidone copolymers, acrylic acid-vinylformamide copolymers and polyacrylates. Especially preferred thickeners are still selected from carbomers. Carbomers are thickening crosslinked polymers of acrylic acid, methacrylic acid, and their salts. Crosslinking can be carried out by employing polyfunctional compounds such as polyalkylene ethers of polysaccharides or polyalcohols, for example sucrose allyl ether, pentaerythritol allyl ether, and propylene allyl ether. Preferred in the context of the present disclosure are homopolymers of acrylic acid or its salts which are cross-linked with a pentaerythritol allyl ether, a sucrose allyl ether or a propylene allyl ether. A thickening agent usable in the context of the present disclosure is a copolymer of C10-30-alkyl acrylate, acrylic acid, methacrylic acid and their esters, which is cross-linked with a sucrose allyl ether or a pentaerythritol allyl ether. Thickeners based on carbomers are the products available under the trade name Carbopol® (BF Goodrich, Ohio, USA) such as Carbopol® 934, Carbopol® 940, Carbopol® 941, Carbopol® 971, Carbopol® 974, Carbopol® EZ2, Carbopol® ETD 2001, Carbopol® ETD 2020, Carbopol® ETD 2050, Carbopol® ultrez 10, Carbopol® ultrez 20, or Carbopol® ultrez 21.

Furthermore, lipophilic thickeners can be used to thicken the antiperspirant cosmetic products as contemplated herein. The preferred lipophilic thickeners as contemplated herein are selected from hydrophobic clay minerals, bentonites, hectorites, pyrogenic silicas and their derivatives.

It may be advantageous to add to the preparations as contemplated herein at least one chelating agent in a total amount of from about 0.01 to about 3.0% by weight, preferably of from about 0.02 to about 1.0% by weight, in particular of from about 0.05 to about 0.1% by weight, based on the total weight of the antiperspirant . In the context of the present disclosure, preferred chelating agents are selected from the group of B-alaninediacetic acid, cyclodextrin, diethylenetriaminepentamethylenephosphonic acid, sodium, potassium, calcium disodium, ammonium and triethanolamine salts of ethylenediaminetetraacetic acid (EDTA), etidronic acid, hydroxyethylethylenediaminetetraacetic acid (HEDTA) and its sodium salts, sodium salts of nitrilotriacetic acid (NTA), diethylenetriaminepentaacetic acid, phytic acid, hydroxypropylcyclodextrin, methylcyclodextrin Aminotrimethylenepho sphonate-pentasodium, ethylenediaminetetramethylenephosphonate-pentasodium, diethylenetriamine-pentaacetate-pentasodium, pentasodium triphosphate, potassium EDTMP, sodium EDTMP, sodium dihydroxyethylglycinate, sodium phytate, sodium polydimethylglycinophenolsulfonate, Tetrahydroxyethylethylenediamine, tetrahydroxypropylethylenediamine, tetrapotassium etidronate, tetranatrium etidronate, tetrasodium iminodisuccinate, trisodium ethylenediamine disuccinate, tetrasodium N,N-bis(carboxymethyl)glutamate, and tetras odium DL- alanine-N,N-diacetate and desferrioxamine.

In order to further improve the antiperspirant and odor-reducing effect of the compositions as contemplated herein, these compositions contain in a preferred form at least one deodorant active substance in a total amount of from about 0.001 to about 15% by weight, based on the total weight of the cosmetic composition. Insofar as ethanol is used as contemplated herein, it is not considered a deodorant active substance in the context of the present disclosure, but a component of the carrier.

As contemplated herein, the preferred deodorant active ingredients are odor absorbers, deodorizing ion exchangers, germ-inhibiting agents, prebiotically active components as well as enzyme inhibitors or, particularly preferred, combinations of the named active ingredients.

Silicates serve as odor absorbers, which at the same time favourably support the rheological properties of the composition as contemplated herein. As contemplated herein, the most preferred silicates are phyllosilicates, in particular montmorillonite, kaolinite, ilite, beidellite, nontronite, saponite, hectorite, bentonite, smectite, calcium silicates and talc.

Other preferred odor absorbers include zeolites, zinc cicinoleate, cyclodextrins, certain metal oxides, such as aluminium oxide, and chlorophyll.

Other odor absorbers preferred by the present disclosure are selected from pearlite.

As contemplated herein, germ-inhibiting or antimicrobial agents are understood to be those agents which reduce the number of skin germs involved in the formation of odors or inhibit their growth. These germs include various species from the group of staphylococci, the group of corynebacteria, anaerocococci and micrococci.

As contemplated herein, organohalogen compounds as well as organohalides, quaternary ammonium compounds, a number of plant extracts and zinc compounds are preferred as germicidal or antimicrobial agents. These include triclosan, chlorhexidine and chlorhexidine gluconate, 3,4,4′-trichlorocarbanilide, bromochlorophene, dichlorophene, chlorothymol, chloroxylenol, hexachlorophene, dichloro-m-xylenol, dequalinium chloride, domiphene bromide, ammonium phenolsulfonate, benzalkonium halides, benzalkonium cetyl phosphate, benzalkonium saccharinates, benzethonium chloride, cetylpyridinium chloride, laurylpyridinium chloride, laurylisoquinolinium bromide, and methylbenzethonium chloride. Furthermore, phenol, phenoxyethanol, disodium dihydroxyethylsulfosuccinylundecylenate, sodium bicarbonate, zinc lactate, sodium phenolsulfonate and zinc phenolsulfonate, ketoglutaric acid, terpene alcohols such as farnesol, chlorophyllin-copper complexes, a monoalkyl glycerol ether with a branched or linear saturated or unsaturated, optionally hydroxylated C6-C22 alkyl radical, particularly preferably α-(2-ethylhexyl)glycerol ether, commercially available as Sensiva® SC 50 (ex Schülke & Mayr), carboxylic acid esters of mono-, di- and triglycerol (e.g. (e.g. glycerol monolaurate, diglycerol monocaprinate), lantibiotics and plant extracts (e.g. green tea and components of lime blossom oil) can be used.

Further preferred deodorant active substances are selected from so-called prebiotically active components, by which as contemplated herein such components are to be understood which only or at least predominantly inhibit the odor-forming germs of the skin microflora, but not the desired, i.e. the non-odor-forming germs which belong to a healthy skin microflora. Explicit mention must be made here of active substances such as conifer extracts, in particular from the group of Pinaceae, and plant extracts from the group of Sapindaceae, Araliaceae, Lamiaceae and Saxifragaceae, in particular extracts of Picea spp., Paullinia sp., Panax sp., Lamium album or Ribes nigrum, and mixtures of these substances.

Other preferred deodorant active ingredients are selected from the germ-inhibiting perfume oils and the Deosafe® perfume oils available from Symrise®, formerly Haarmann and Reimer.

Other preferred deodorant active substances are selected from silver salts, in particular silver citrate, dihydrogen silver citrate, silver lactate and silver sulphate, soluble complex salts of silver, colloidal silver and silver zeolites.

Enzyme inhibitors include substances which inhibit the enzymes responsible for sweat decomposition, in particular arylsulfatase, β-glucuronidase, aminoacylase, esterases, lipases and/or lipoxigenase, e.g. trialkyl citric acid esters, in particular triethyl citrate, or zinc glycinate.

Preferred antiperspirant compositions as contemplated herein include at least one deodorant active ingredient which is selected from arylsulfatase inhibitors, beta-glucuronidase inhibitors, aminoacylase inhibitors, esterase inhibitors, lipase inhibitors and lipoxigenase inhibitors, α monoalkylglycerol ethers with a branched or linear saturated or unsaturated, optionally hydroxylated C6-C22 alkyl radical, in particular α-(2-ethylhexyl) glycerol ether, phenoxyethanol benzylheptanol, cocamidopropyl PG-dimonium chloride phosphates and butyloctanoic acid, polyglycerol-3-caprylates, antibacterial perfume oils, Deosafe® perfume oils (Deosafe® is a registered trademark of Symrise®, formerly Haarmann & Reimer), prebiotically active components, trialkyl citric acid esters, in particular triethyl citrate, active substances which reduce the number of skin germs from the group of staphylococci, corynebacteria, anaerocococci and micrococci involved in the formation of odors, or inhibit their growth, zinc compounds, in particular zinc phenolsulphonate and zinc cicinoleate, organohalogen compounds, in particular triclosan, chlorhexidine, chlorhexidine gluconate and benzalkonium halides, quaternary ammonium compounds, in particular cetylpyridinium chloride, odor absorbers, in particular silicates and zeolites, sodium bicarbonate, lantibiotics, and mixtures of the aforementioned substances.

Further preferred antiperspirant compositions as contemplated herein include at least one deodorant active substance which is contained in a total amount of from about 0.0001-15% by weight, preferably from about 0.2-7% by weight, particularly preferably from about 0.3-5% by weight and extremely preferably from about 0.4-1.0% by weight, in each case based on the total weight of the active substance of the deodorant active substance or deodorant active substances in the total composition.

The compositions as contemplated herein contain both at least one deodorant and at least one antiperspirant active substance in a further particularly preferred form.

Preferred compositions as contemplated herein further contain at least one water-soluble polyvalent C₂-C₉ alkanol with 2-6 hydroxyl groups and/or at least one water-soluble polyethylene glycol with about 3-50, preferably about 3-20 ethylene oxide units as well as mixtures thereof to further improve the stability of the compositions. Preferably these components are selected from 1,2-propylene glycol, 2-methyl-1,3-propanediol, glycerol, 1,2-butylene glycol, 1,3-butylene glycol, 1,4-butylene glycol, pentylene glycols such as 1,2-pentanediol and 1,5-pentanediol, hexanediols such as 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol and mixtures of the above substances. Suitable water-soluble polyethylene glycols are selected from PEG-3, PEG-4, PEG-6, PEG-7, PEG-8, PEG-9, PEG-10, PEG-12, PEG-14, PEG-16, PEG-18 and PEG-20 and mixtures thereof, wherein PEG-3 to PEG-8 are preferred. Preferably propylene glycol, dipropoylene glycol or a mixture of these is contained, more preferably only propylene glycol. Preferably they are contained in a total quantity of from about 0.1 to about 50% by weight, more preferably from about 0.5 to about 10 or from about 1 to about 5% by weight.

According to a further embodiment of the present disclosure, the antiperspirant cosmetic products further contain at least one skin-cooling active substance. Skin-cooling active substances suitable as contemplated herein are for example menthol, isopulegol as well as menthol derivatives, e.g. menthyl lactate, menthyl glycolate, menthyl ethyl oxamate, menthyl pyrrolidone carboxylic acid, menthyl methyl ether, menthoxypropanediol, menthonglycerol acetal (9-methyl-6-(1-methylethyl)-1,4-dioxaspiro (4 ,5)dec an-2-methanol), monomenthyl succinate, 2-hydroxymethyl-3,5,5-trimethylcyclohexanol and 5-methyl-2-(1-methylethyl)cyclohexyl-N-ethyloxamate. Preferred skin cooling agents are menthol, isopulegol, menthyl lactate, menthoxypropanediol, menthyl pyrrolidone carboxylic acid and 5-methyl-2-(1-methylethyl)cyclohexyl-N-ethyloxamate as well as mixtures of these substances, in particular mixtures of menthol and menthyl lactate, menthol, menthol glycolate and menthyl lactate, menthol and menthoxypropanediol or menthol and isopulegol.

Finally, another object of the present disclosure is a non-therapeutic cosmetic method for preventing and/or reducing perspiration of the body, wherein an antiperspirant cosmetic agent as contemplated herein is applied to the skin, in particular to the skin of the armpits, and remains on the skin of the armpits for at least about 1 hour, preferably for at least about 2 hours, preferably for at least about 4 hours, in particular for at least about 6 hours.

With regard to the method, what has been said about the antiperspirant cosmetic products applies mutatis mutandis.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An antiperspirant cosmetic composition which comprises in an aqueous cosmetic carrier based on a total weight of the cosmetic composition:
a) at least one poly(vinylamine-vinylformamide) copolymer having a molecular weight of from about 300 to about 20,000 g/mol in a total amount of from about 0.1 to about 20% by weight,
b) at least one emulsifier in a total amount of from about 0.05 to about 10% by weight, and
c) at least one oil component in a total amount of from about 0.05 to about 50% by weight,
where
the antiperspirant cosmetic composition has a pH value of from about 2.5 to about 7.5 and the poly(vinylamine-vinylformamide) copolymer is neutralized or polycationic,
the antiperspirant cosmetic composition comprises at least one anion selected from the group of chloride, phosphates and sulphates, and
the antiperspirant cosmetic composition does not contain halides and/or hydroxyhalides of aluminium and/or zirconium.

2. The antiperspirant cosmetic composition according to claim 1, wherein the antiperspirant composition comprises from about 0.5 to about 10% by weight of the poly(vinylamine-vinylformamide) copolymer.

3. The antiperspirant cosmetic composition according to claim 1, wherein the poly(vinylamine-vinylformamide) copolymer comprises from about 10- to about 99 mol % of vinylamine monomer units.

4. The antiperspirant cosmetic composition according to claim 1, wherein the poly(vinylamine-vinylformamide) copolymer has a weight average molecular weight of from about 500 to about 6000 g/mol.

5. The antiperspirant cosmetic composition according to claim 1, wherein the poly(vinylamine-vinylformamide) copolymer is adsorbed on solids, and wherein the poly(vinylamine-vinylformamide) copolymer is used in the form of particles which fall about 100% through a sieve of mesh size about 200 μm.

6. The antiperspirant cosmetic composition according to claim 1, wherein at least about 10% by weight of the anions contained are chloride ions.

7. The antiperspirant cosmetic composition according to claim 1, wherein the antiperspirant cosmetic composition comprises the at least one emulsifier in a total amount of from about 0.2 to about 5.0% by weight, based on the total weight of the cosmetic composition.

8. The antiperspirant cosmetic composition according to claim 1, wherein the antiperspirant cosmetic composition comprises at least one thickening agent.

9. The antiperspirant cosmetic composition according to claim 1, wherein the antiperspirant cosmetic composition comprises at least one further anion.

10. A non-therapeutic cosmetic method for preventing and/or reducing body odor and/or perspiration of the body, comprising:
applying a cosmetic agent according to claim 1 to the skin, and leaving the cosmetic agent on the skin for at least 1 hour.

11. The antiperspirant cosmetic composition according to claim 9, wherein the at least one further anion is selected from citrate, lactate, tartrate, malate, other anions of organic acids, and combinations thereof.

12. The antiperspirant cosmetic composition according to claim 1, wherein the antiperspirant cosmetic composition comprises the poly(vinylamine-vinylformamide) copolymer in an amount of from about 2.0 to about 5.0 by weight, based on the total weight of the antiperspirant cosmetic composition.

13. The antiperspirant cosmetic composition according to claim 1, wherein the poly(vinylamine-vinylformamide) copolymer comprises at least 95% of vinylamine monomer units.

14. The antiperspirant cosmetic composition according to claim 1, wherein the poly(vinylamine-vinylformamide) copolymer has a weight average molecular weight of from about 1,000 to about 3,000 g/mol.

15. The antiperspirant cosmetic composition according to claim 1, wherein the poly(vinylamine-vinylformamide) copolymer is adsorbed on solids, and wherein the poly(vinylamine-vinylformamide) copolymer is used in the form of particles which fall about 100% through a sieve of mesh size about 100 μm.

16. The antiperspirant cosmetic composition according to claim 15, wherein at least 30% by weight of the particles remain on a sieve of mesh size 10 μm.

17. The antiperspirant cosmetic composition according to claim 1, wherein at least about 95.0% by weight of the anions contained are chloride ions.

18. The antiperspirant cosmetic composition according to claim 1, wherein the antiperspirant cosmetic composition comprises the at least one emulsifier in a total amount of from about 1.5 to about 3.0% by weight, based on the total weight of the cosmetic composition.

19. The antiperspirant cosmetic composition according to claim 1, wherein the antiperspirant cosmetic composition comprises the poly(vinylamine-vinylformamide) copolymer in an amount of from about 1.0 to about 7.0 by weight, based on the total weight of the antiperspirant cosmetic composition.

20. The antiperspirant cosmetic composition according to claim 1, wherein the antiperspirant cosmetic composition comprises the poly(vinylamine-vinylformamide) copolymer in an amount of from about 2.0 to about 5.0 by weight, based on the total weight of the antiperspirant cosmetic composition, the antiperspirant cosmetic composition comprises the at least one emulsifier in a total amount of from about 1.5 to about 3.0% by weight, based on the total weight of the antiperspirant cosmetic composition, wherein the poly(vinylamine-vinylformamide) copolymer comprises at least 95% of vinylamine monomer units, wherein the poly(vinylamine-vinylformamide) copolymer has a weight average molecular weight of from about 1,000 to about 3,000 g/mol, and wherein the antiperspirant cosmetic composition comprises the at least one emulsifier in a total amount of from about 1.5 to about 3.0% by weight, based on the total weight of the cosmetic composition.
